(19) United States
(12) Patent Application Publication
(10) Pub. No.: US 2022/0168240 A1
(43) Pub. Date: Jun. 2, 2022

(54) CANCER THERAPY BY INTRATUMORAL INJECTION OF A CHEMOTHERAPEUTIC AGENT IN COMBINATION WITH A BIOACTIVE IMMUNOSTIMULATORY AGENT

(71) Applicant: SYBLEU INC., CHEYENNE, WY (US)

(72) Inventor: David KOOS, La Mesa, CA (US)

(73) Assignee: SYBLEU INC., CHEYENNE, WY (US)

(21) Appl. No.: 17/541,088

(22) Filed: Dec. 2, 2021

Related U.S. Application Data
(60) Provisional application No. 63/120,673, filed on Dec. 2, 2020.

(51) Int. Cl. A61K 31/135; A61K 45/06

(52) U.S. Cl. CPC A61K 31/135; A61K 45/06

(57) ABSTRACT

The teachings herein are directed to methods of treating a tumor comprising identifying a patient having a tumor; and administering a combination of a chemotherapeutic agent and an immunomodulatory agent for cancer therapy into the tumor in an amount sufficient to treat the tumor.

### CROSS-REFERENCE TO RELATED APPLICATIONS

This application claims the benefit of priority to U.S. Provisional Application No. 63/120,673, filed on Dec. 2, 2020, the contents of which are incorporated herein in its entirety.

### FIELD OF THE INVENTION

The methods herein are in the field of cancer therapeutics.

### BACKGROUND

Current cancer treatment modalities include the use of chemotherapeutic agents, radiation, surgical removal of tumor tissue and more recently the use of biologically active proteins or factors such as GM-CSF or IL-12 for the stimulation of anti-cancer immune responses. Chemotherapeutic agents are often delivered systemically through intravenous or oral administration whereas the protein factors are often administered by intravenous infusions. In contrast to systemic administration, the development of sustained release formulations (PLGA) has been reported (1,2) which can be used for localized delivery such as directly injecting such agents into tumors.

### SUMMARY

Preferred embodiments are directed to methods of treating a tumor comprising: identifying a patient having a tumor; and administering a combination of a chemotherapeutic agent and an immunomodulatory agent for cancer therapy into the tumor in an amount sufficient to treat the tumor.

Preferred methods are directed to embodiments wherein the chemotherapeutic agent is in a biodegradable material.

Preferred methods are directed to embodiments wherein the chemotherapeutic agent is in a biocompatible material.

Preferred methods are directed to embodiments wherein the biocompatible material is a PLGA microsphere.

Preferred methods are directed to embodiments wherein the chemotherapeutic agent is AQ4N.

Preferred methods are directed to embodiments wherein the chemotherapeutic agent is AQ4 1,4-Bis[[2-dimethylamino)ethyl]amino]-5,8-dihydroxyanthracene-9,10 dione.

Preferred methods are directed to embodiments wherein the immunomodulatory agent is selected from the group consisting of: TNF-alpha, GM-CSF, Claim IL-4, IL-2, IL-12, IL-23, DNA, RNA, Nucleic Acids, and CD-40 Ligand.

Preferred methods are directed to embodiments wherein the chemotherapeutic agent is a topoisomerase inhibitor.

Preferred methods are directed to embodiments the topoisomerase inhibitor is Mitoxantrone.

Preferred methods are directed to embodiments the immunomodulatory agent is selected from the group consisting of: TNF-alpha, GM-CSF, Claim IL-4, IL-2, IL-12, IL-23, DNA, RNA, Nucleic Acids, and CD-40 Ligand.

Preferred methods are directed to embodiments the chemotherapeutic agent and an immunomodulatory agent are administered together simultaneously.

### DETAILED DESCRIPTION

The teachings herein are directed to the simultaneous, intratumoral injection of one or more chemotherapeutic agents in combination with one or more immunomodulatory agents in sustained release formulations. Additionally, the chemotherapeutic agent and the immunomodulatory agent can be administered within an hour, 30 minutes, 15 minutes, or 5 minutes. The chemotherapeutic agent is administered for the purpose of directly killing the tumor cells and for the release of antigens while the immunomodulatory protein or factor is administered to stimulate the antigenic response of the host to the antigens.

An example of this idea would be to make PLGA micro spheres containing a chemotherapeutic agent as listed in Table 1. The compounds that inhibit topoisomerases such as Mitoxantrone are of interest since they induce apoptosis or programmed cell death. Two potential compounds that are not listed but related to Mitoxantrone are AQ4N a non-toxic pro-drug and its toxic metabolite AQ4 1,4-Bis[[2-dimethylamino)ethyl]amino]-5,8-dihydroxyanthracene-9,10 dione. AQ4 is generated mainly in hypoxic tumor tissue. They are also believed to induce necrosis. In either case, the cytotoxic effects of these agents result in antigen release which aids in the immune recognition of the specific tumor antigens.

The cytokines, growth factors or agents of interest would be those that enhance or stimulate the immune response. These would include GM-CSF, IL-4, IL-2, IL-12, IL-23, DNA, RNA, Nucleic Acids, CD40 Ligand, TNF-alpha.

TABLE 1
Intracellular chemotherapeutic agents/antineoplastic agents (L01)

| SPs/MIs | Block microtubule | Vinca alkaloids (Vinblastine, Vincristine, | |
|---|---|---|---|
| (M phase) | assembly | Vinflunine, Vindesine, Vinorelbine) | |
| | Block microtubule | Taxanes (Docetaxel, Larotaxel, Ortataxel, | |
| | disassembly | Paclitaxel, Tesetaxel) | |
| | | Epothilones: (Ixabepilone) | |
| DNA replication | DNA precursors/ | Folic acid | dihydrofolate reductase |
| inhibitor | antimetabolites | | inhibitor |
| | (S phase) | | (Aminopterin, Methotrexate, |
| | | | Pemetrexed) |
| | | | thymidylate synthase inhibitor |
| | | | (Raltitrexed, Pemetrexed) |
| | | Purine | adenosine deaminase inhibitor |
| | | | (Pentostatin) |
| | | | halogenated/ribonucleotide reductase |
| | | | inhibitors |
| | | | (Cladribine, Clofarabine, |
| | | | Fludarabine) |
| | | | thiopurine |
| | | | (Thioguanine, Mercaptopurine) |
| | | Pyrimidine | thymidylate synthase inhibitor |
| | | | (Fluorouracil, Capecitabine, |
| | | | Tegafur, Carmofur, Floxuridine) |
| | | | DNA polymerase inhibitor |
| | | | (Cytarabine) |
| | | | ribonucleotide reductase inhibitor |
| | | | (Gemcitabine) |
| | | | hypomethylating agent |
| | | | (Azacitidine, Decitabine) |
| | | Deoxyribo- | ribonucleotide reductase inhibitor |
| | | nucleotide | (Hydroxyurea) |
| DNA replication | Topoisomerase | I | Camptotheca |
| inhibitor | inhibitors | | (Camptothecin, Topotecan, |
| | | | Irinotecan, Rubitecan, Belotecan) |
| | | II | Podophyllum |
| | | | (Etoposide, Teniposide) |
| | | II + Intercalation | Anthracyclines |
| | | | (Aclarubicin, Daunorubicin, |
| | | | Doxorubicin, Epirubicin, |
| | | | Idarubicin, Amrubicin, Pirarubicin, |
| | | | Valrubicin, Zorubicin) |
| | | | Anthracenediones |
| | | | (Mitoxantrone, Pixantrone) |
| | Crosslinking of | Alkylating | Nitrogen mustards |
| | DNA | | Mechlorethamine, Cyclophosphamide, |
| | (CCNS) | | (Ifosfamide, Trofosfamide) |
| | | | Chlorambucil (Melphalan, |
| | | | Prednimustine) |
| | | | Bendamustine, Uramustine, |
| | | | Estramustine |
| | | | Nitrosoureas |
| | | | Carmustine, Lomustine (Semustine) |
| | | | Fotemustine, Nimustine, Ranimustine, |
| | | | Streptozocin |
| | | | Alkyl sulfonates |
| | | | Busulfan (Mannosulfan, Treosulfan) |
| | | | Aziridines |
| | | | Carboquone, ThioTEPA, Traziquone |
| | | | Triethylenemelamine |
| | | Alkylating-like | Platinum |
| | | | (Carboplatin, Cisplatin, Nedaplatin, |
| | | | Oxaliplatin,Triplatin, tetranitrate, |
| | | | Satraplatin) |
| | | Nonclassical | Hyrdrazines Procarbazine |
| | | | Triazenes |
| | | | (Dacarbazine, Temozolomide) |
| | | | Altretamine, Mitobronitol |
| | | Intercalation | Streptomyces (Actinomycin, |
| | | | Bleomycin, Mitomycin, Plicamycin |

| Photosensitizers/ | Aminolevulinic acid/Methyl aminolevulinate, Efaproxiral | |
|---|---|---|
| PDT | Porphyrin derivatives (Porfimer sodium, Talaporfin, Temoporfin, Verteporfin) | |
| Other | Enzyme | FI (Tipifarnib) |
| | inhibitors | CDK inhibitors (Alvocidib, Seliciclib) |
| | | PrI |
| | | (Bortezomib) |
| | | PhI (Anagrelide) |
| | | IMPDI (Tiazofurine) |
| | | LI (Masoprocol) |
| | | PARP inhibitor (Olaparib) |
| | Receptor | ERA |
| | antagonists | (Atrasentan) |
| | | retinoid X receptor (Bexarotene) |
| | | sex steroid (Testolactone) |
| | Other/ungrouped | Amsacrine, Trabectedin |
| | | retinoids (Alitretinoin, Tretinoin) |
| | | Arsenic trioxide |
| | | asparagine depleter (Asparaginase/Pegaspargase) |
| | | Celecoxib, Demecolcine, Eleclomol, Elsamitrucin, |
| | | Etoglucid, |
| | | Lonidamine, Lucanthone, Mitoguazone, Mitotane, |
| | | Oblimersen, Temsirolimus, Verinostat |

### REFERENCES:

1. Egilmez N K, Jong Y S, Sabel M S, Jacob J S, Mathiowitz E, Bankert R B. 2000. In situ tumor vaccination with Interleukin-12-encapsulated biodegradable microspheres: Induction of tumor regression and potent antitumor immunity. Cancer Research, 60:3832-3837.

2. Radulescu D, Schwade N, Wawro D. 2003. Uniform Paclitaxel-loaded biodegradable microspheres manufactured by ink-jet technology. Proc Recent Adv in Drug Delivery Sys, March 2003, pages 1-5.

1. A method of treating a tumor comprising:
identifying a patient having a tumor; and
administering a combination of a chemotherapeutic agent and an immunomodulatory agent for cancer therapy into the tumor in an amount sufficient to treat the tumor.

2. The method of claim 1, wherein the chemotherapeutic agent is in a biodegradable material.

3. The method of claim 1, wherein the chemotherapeutic agent is in a biocompatible material.

4. The method of claim 3, wherein the biocompatible material is a PLGA microsphere.

5. The method of claim 1, wherein the chemotherapeutic agent is AQ4N.

6. The method of claim 1, wherein the chemotherapeutic agent is AQ4 1,4-Bis[[2-dimethylamino)ethyl]amino]-5,8-dihydroxyanthracene-9,10 dione.

7. The method of claim 5, wherein the immunomodulatory agent is selected from the group consisting of: TNF-alpha, GM-CSF, Claim IL-4, IL-2, IL-12, IL-23, DNA, RNA, Nucleic Acids, and CD-40 Ligand.

8. The method of claim 1, wherein the chemotherapeutic agent is a topoisomerase inhibitor.

9. The method of claim 8, wherein the topoisomerase inhibitor is Mitoxantrone.

10. The method of claim 9, wherein the immunomodulatory agent is selected from the group consisting of: TNF-alpha, GM-CSF, Claim IL-4, IL-2, IL-12, IL-23, DNA, RNA, Nucleic Acids, and CD-40 Ligand.

11. The method of claim 1, wherein the chemotherapeutic agent and an immunomodulatory agent are administered together simultaneously.
